(19) United States
(12) Patent Application Publication
(10) Pub. No.: US 2016/0081901 A1
(43) Pub. Date: Mar. 24, 2016

(54) COMPOSITIONS AND METHODS FOR HAIR GROWTH

(71) Applicants: Junjie YANG, Wuhan (CN); WUHAN OPTICS VALLEY BRIDGEBIOMED INTERNATIONAL CORPORATION, Wuhan (CN)

(72) Inventors: JUNJIE YANG, Wuhan (CN); LINYA LI, Wuhan (CN); BEILI ZHU, Wuhan (CN)

(21) Appl. No.: 14/891,648

(22) PCT Filed: Apr. 30, 2014
(86) PCT No.: PCT/US2014/036270

§ 371 Date: Nov. 16, 2015

(30) Foreign Application Priority Data
May 16, 2013 (CN) 201310181805

(51) Int. Cl. A61K 8/49; C07D 403/04; A61Q 7/00; C07D 403/10

(52) U.S. Cl. CPC A61K 8/4946; C07D 403/10; C07D 403/04; A61Q 7/00

(57) ABSTRACT

The present invention provides compositions comprising a bradykinin receptor B2 agonist or an angiotensin II receptor (ATIR) antagonist, and methods using such compositions to increase/promote hair growth or reduce/delay hair loss.

### FIELD

The invention relates in general to the cosmetic and pharmaceutical fields, and more particularly to compositions and methods for increasing/promoting hair growth or reducing/delaying hair loss.

### BACKGROUND

Bradykinin (BK) is a 9 amino acid peptide which can cause vasodilation and therefore lower blood pressure. BK binds to BK receptors which belong to the G-protein-coupled receptors (GPCRs). Two different subtypes BK receptors designated B1 and B2 have been identified. B2 receptors are constitutively expressed in a variety of healthy cells and they form a complex with angiotensin converting enzyme (ACE), and thus thought to play a role in cross-talk between the renin-angiotensin system (RAS) and the kinin-kallikrein system (KKS). B1 receptors are constitutively expressed at much lower level, but upregulated by tissue injury, inflammation and cytokines, and is presumed to play a role in chronic pain. B2 receptors are related to a burst of prostaglandin (PG) release, whereas B1 receptors mediate a delayed prostaglandin response. BK agonists that bind to B2 receptors can activate PhospholipaseA2, convert phospholipids to arachidonic acid, which is converted to PGs by Cyclooxygenase (1, 2). In eyes, these PGs production can increase aqueous humour outflow to lowing intraocular pressure (IOP). It has also been shown that BK B2 receptors are stimulated in the pathogenesis of inflammation, pain and tissue injury.

Undesired hair loss is a common problem for many men and women. There have been continuous efforts in the cosmetic and pharmaceutical industry searching for substances effective in reducing/delaying hair loss or increasing/promoting hair growth. BK antagonists inhibiting BK receptor signaling have been indicated in treating hair loss, e.g., see U.S. Pat. No. 6,468,972.

### SUMMARY OF THE INVENTION

The present invention provides a topical composition comprising at least one active ingredient selected from the group consisting of a bradykinin (BK) B2 receptor agonist and an angiotensin II receptor (AT1R) antagonist.

According to certain embodiments, the active ingredient has a concentration of from about 0.01% to about 10% by weight.

For example, the active ingredient may have a concentration of from about 0.03% to about 3% by weight, or the active ingredient may have a concentration of about 0.03% by weight, or the active ingredient may have a concentration of about 0.3% by weight, or the active ingredient may have a concentration of about 3% by weight.

The active ingredient may be a BK B2 receptor agonist according to certain embodiments.

The active ingredient may be an angiotensin II receptor (AT1R) antagonist according to certain embodiments.

The active ingredient may be a BK B2 receptor agonist and an angiotensin II receptor (AT1R) antagonist according to certain embodiments.

According to certain embodiments, the active ingredient is a non-peptide compound.

According to certain embodiments, the active ingredient is Losartan, or Candesartan, or Telmisartan.

According to certain embodiments, the active ingredient is 2-ethoxy-1-[[2′-(1H-tetrazol-5-yl)biphenyl-4-yl]methyl]benzlimidazole-7-carboxylic acid.

According to certain embodiments, the active ingredient is 1-(cyclohexyloxycarbonyloxy)ethyl-2-ethoxy-1-[[2′-(1H-tetrazol-5-yl)biphenyl-4-yl]methyl]benzimidazole-7-carboxylate.

According to certain embodiments, the active ingredient is 2-ethoxy-1-[[2′-(2,5-dihiydro-5-oxo-1,2,4-ox-adiazol-3-yl)biphenyl-4-yl]methyl]benzimidazole-7-carboxylic acid.

The topical composition may further comprise at least a pharmaceutical carrier selected from the group consisting of transdermal permeation enhancer, transdermal absorption promoting agent, water, solvent, preservative, surfactant, and a pH balancer, according to certain embodiments of the invention.

The transdermal permeation enhancer may be propylene glycol, Azone, or a combination thereof according to certain embodiments. The propylene glycol or Azone may have a concentration of 2% by weight.

The topical composition may comprise alcohol and/or a PBS solution according to certain embodiments. The PBS solution may have a pH at from about 6.5 to about 7.8.

The topical composition may be in the form of a gel, liniment, cream or ointment according to certain embodiments.

The present invention provides use of the topical composition in the preparation of a medicament for reducing or delaying hair loss according to certain embodiments. The hair loss may be caused by androgenetic alopecia, or seborrheic alopecia.

The present invention provides use of the topical composition in the preparation of a medicament for increasing or promoting hair growth according to certain embodiments. The hair growth may be eye brow or eye lash growth.

According to certain embodiments, the hair loss or growth may be related to PG E/F2a release.

The present invention provides a method of reducing or delaying hair loss in a subject comprising administering the topical composition to the subject in an amount effective to reduce or delay hair loss, according to certain embodiments. The hair loss may be caused by androgenetic alopecia, or seborrheic alopecia.

The present invention provides a method of increasing or promoting hair growth in a subject, comprising administering the topical composition to the subject in an amount effective to increase or promote hair growth. The hair growth may be eye brow or eye lash growth.

The hair loss or growth is related to PG E/F2a release according to certain embodiments.

According to certain embodiments, the daily therapeutic dose of the active ingredient administered is from about 0.01 mg to about 500 mg. For example, the daily therapeutic dose of the active ingredient administered may be from about 0.1 mg to about 300 mg, or from about 1 mg to about 250 mg.

The present invention provides a kit comprising the topical composition according to certain embodiments.

The present invention provides a computer modeling structure identifying a series of BK B2 receptor agonist binding sites of within BK B2 receptor according to certain embodiments. The binding sites may be presented in 2-dimentions or 3-dimentions. The binding sites may be identified using molecular modeling. The molecular modeling may be based on one or more BK B2 receptor agonists selected from the group consisting of Losartan, Candesartan, and Telmisartan.

According to certain embodiments, the binding sites are within carboxylic acid group and tetrazole ring of Losartan and optionally interact with binding pocket border.

According to certain embodiments, the binding sites are selected from the group consisting of S87, S88, C89, T90, A92, E93 of TM2, T264, E265, R267, A268 of TM6, and Y347, R338, F339, V350 of C-term loop of BK B2 receptor, respectively.

According to certain embodiments, the molecular modeling indicates interaction between the carboxylic acid group and tetrazole ring of Candesartan with binding pocket containing S87, S88, C89, T90, A92, E93 of TM2, D154, R155, A158, R167 of TM3, T264, E265, R267, A268, L271, V272 of TM6, Y347, and R338 of C-term loop.

### BRIEF DESCRIPTION OF THE DRAWINGS

FIG. 1 is a simplified, schematic illustration showing PFSC code of Losartan binding sites, which are related to the present disclosure.

FIG. 2 is a simplified, schematic illustration showing PFSC code of Candesartan binding sites, which are related to the present disclosure.

FIG. 3 is a simplified, schematic illustration Candesartan assay by HPLC, which are related to the present disclosure.

FIG. 4 is a simplified schematic illustration showing the hair growth condition before treatment (FIG. 4A), after treatment within one month (FIG. 4B), and with treatment beyond 2 months (FIG. 4C) in accordance with yet another embodiment of the present disclosure.

### DETAILED DESCRIPTION

The present invention provides compositions and methods for effective treatment of hair loss and promotion of hair growth. The inventors of the present invention discovered that angiotensin II receptor antagonists could stimulate bradykinin receptor B2 and therefore trigger secretion of endogenous prostaglandins (PG) F-2a; local microenvironment favoring hair growth could be improved by PG receptor pathway. Meanwhile, angiotensin II receptor antagonists could efficiently block the action of angiotensin II, and improve local blood flow, thus promote hair growth. The angiotensin II receptor antagonists and/or bradykinin receptor B2 agonists described herein is dual-target innovative drug with classical structure and new mechanism; which makes it a novel dual-target drug for hair growth promotion.

This invention further provides a method of developing novel drug against hair loss. Since angiotensin II receptor antagonists described herein could bind multiple sites and simultaneously improve local blood flow through vasodilation as well as act as BK B2 receptor agonist, hair growth could be promoted through regulating the amount and proportion of PG and PG subtypes within local microenvironment.

This invention also provides theoretical basis for multi-target drug development. Moreover, drug could be directly applied by topical application to the treatment sites, and take effect locally, thus reduce the side effect of systemic drug application. The present invention relates to a series of compounds and pharmaceutical compositions comprising one or more of these compounds and methods of administering them for the treatment of PG D/PG F2a ratio related hair loss by increasing PG-F 2a release.

Angiotensin receptor antagonists and/or BK B2 receptor agonists may be used in the compositions and methods according to some embodiments of the present invention.

The angiotensin receptors are a class of G protein-coupled receptors, and there are two subtypes: Angiotensin II receptor type 1 (AT1) and Angiotensin II receptor type 2 (AT2). The AT1 receptor regulates aldosterone secretion, vasoconstriction, and excites beta adrenergic nerves while promotes growth. While the AT2 receptor inhibits hyperplasia and dilates blood vessels.

Angiotensin receptor blockers (ARBs), also known as angiotensin II receptor (AT1R) antagonists, block the activation of AT1 receptors and inhibit vasoconstriction, as well as decrease the peripheral vascular resistance and thus are indicated for the treatment of hypertension. The ARBs specifically bind AT1, and simultaneously avoid dry cough and/or angioedema while do not inhibit the breakdown of bradykinin (BK). Nowadays, losartan, candesartan, telmisartan are some representatives of AT1 receptor antagonist drugs.

Known Angiotensin II Receptor Blockers (ARBs) include, but are not limited to, the following examples:

| Generic | Brand | |
|---|---|---|
| Name | Name | Systematic (IUPAC) name |
| candesartan | Atacand | 2-ethoxy-1-({4-[2-(2H-1,2,3,4- |
| | | tetrazol-5- |
| | | yl)phenyl]phenyl}methyl)-1H- |
| | | 1,3-benzodiazole-7-carboxylic |
| | | acid |
| eprosartan | Teveten | 4-({2-Butyl-5-[2-carboxy-2- |
| | | (thiophen-2-ylmethyl)eth-1-en- |
| | | 1-yl]-1H-imidazol-1- |
| | | yl}methyl)benzoic acid |
| irbesartan | Avapro | 2-butyl-3-({4-[2-(2H-1,2,3,4- |
| | | tetrazol-5- |
| | | yl)phenyl]phenyl}methyl)-1,3- |
| | | diazaspiro[4.4]non-1-en-4-one |
| losartan | Cozaar | (2-butyl-4-chloro-1-{[2′-(1H- |
| | | tetrazol-5-yl)biphenyl-4- |
| | | yl]methyl}-1H-imidazol-5- |
| | | yl)methanol |
| olmesartan | Benicar | (5-methyl-2-oxo-2H-1,3-dioxol- |
| | | 4-yl)methyl 4-(2- |
| | | hydroxypropan-2-yl)-2-propyl-1- |
| | | ({4-[2-(2H-1,2,3,4-tetrazol-5- |
| | | yl)phenyl]phenyl}methyl)-1H- |
| | | imidazole-5-carboxylate |
| telmisartan | Micardis | 2-(4-{[4-methyl-6-(1-methyl-1H- |
| | | 1,3-benzodiazol-2-yl)-2-propyl- |
| | | 1H-1,3-benzodiazol-1- |
| | | yl]methyl}phenyl)benzoic acid |
| valsartan | Diovan | (S)-3-methyl-2-(N-{[2′-(2H- |
| | | 1,2,3,4-tetrazol-5-yl)biphenyl-4- |
| | | yl]methyl}pentanamido)butanoic |
| | | acid |

Losartan, Candesartan and Telmisartan, are all angiotensin II receptor antagonist drug whose main use are to treat hypertension. Losartan was the first AT1R antagonist drug on market, while candesartan is a relatively novel antihypertensive agent of the AT1R antagonist drug. Telmisartan, of all the commercially available ARBs, has the longest half-life of about 24 h. Their administrations result in a decrease in total peripheral resistance (afterload) and cardiac venous return (preload).

Some or all angiotensin II receptor antagonists may also be BK B2 receptor agonists.

All of the physiological effects of angiotensin II, including stimulation of release of aldosterone, are antagonized in the presence of Losartan, or Candesartan, or Telmisartan. Reduction in blood pressure occurs independently of the status of the renin-angiotensin system.

The angiotensin II type 1 receptor (AT1R) blockers (ARBs) Losartan, Candesartan and Telmisartan have cardioprotective effects during ischemia-reperfusion injury and inhibits reperfusion arrhythmias effects that go beyond the benefits of lowering blood pressure. All of them meanwhile activated the BK B2 receptor, working as a partial agonist compared to the endogenous ligand bradykinin. This effect was blocked by the B2R antagonist HOE 140. Losartan, Candesartan, Telmisartan mimicked the ability of bradykinin to increase the release of PGs.

Prostaglandins (PG) are a group of lipid compounds and have important functions in the animal body. Prostaglandin was first isolated from seminal fluid,[2][3] and it was believed to be part of the prostatic secretions, therefore it was named prostaglandins. Now it has been proved that prostaglandins are mainly produced by the seminal vesicles, and could be produced by cells from most tissues and organs.

The prostaglandins are produced in vivo from arachidonic acids, and every prostaglandin contains 20 carbon atoms, including a 5-carbon ring. There're different types of prostaglandins, i.e. A, B, C, D, E, F, G, H, I, since their structures differ. Different types of prostaglandins have different functions, e.g. Prostaglandin E could diastole bronchial smooth muscle, thus decrease airway resistance; while Prostaglandin F has the opposite functionalities. The half-life of prostaglandins are very short (usually from 1-2 minutes), and all the prostaglandins except Prostaglandin I degrade quickly through liver and lung. Unlike typical hormones affecting a discrete site through the circulatory system, prostaglandins are produced and secreted locally, and modulate the activities at their sites of secretion.

Previous research indicates that prostaglandin F-2a promotes hair growth, while prostaglandin D2 inhibits hair growth; which suggests prostaglandins are crucial in local endocrine modulation as well as hair growth.

PGE2 is known to act synergistically with PGF2alpha, and hence the influence of PGE2 was also examined. PGE2 did not induce distinct telogen-to-anagen conversion, but showed moderate growth stimulatory effects on early anagen hair follicles.

Kinins, Bradykinin (BK) agonists, must first bind to cell surface receptors in order to exert their biological effects. BK receptors belong to G-protein-coupled receptors (GPCRs). There are at least two different subtypes, B1 and B2 receptors. It was reported that the two receptor subtypes are linked to different signal transduction mechanisms. BK agonists that can bind to B2 receptor activate PhospholipaseA2. This enzyme converts phospholipids to arachidonic acid. Cyclooxygenase 1&2 in turn catalyze the formation of PGs from this fatty acid. In eyes, the PGs production can increase Aqueous humor outflow, leading to lower intraocular pressure (IOP).

Angiotensin AT1 receptor antagonists act also partly through kinins. Direct activation of the kinin B2 receptor by pharmacological agonists might provide higher therapeutic benefit than existing kinin-potentiating drugs. Local application of B2 receptor agonists stimulates the endogenous prostaglandin secretion; similarly Losartan, Candesartan and Telmisartan could trigger total PG and Subtype PG release via Bradykinin B2 receptor and modulates the ratio between prostaglandin F-2a and prostaglandin D2, thus promotes hair growth.

Latanoprost, analogues of prostaglandin F2alpha (PGF2alpha), used clinically in the treatment of glaucoma, induces growth of lashes and ancillary hairs around the eyelids. The increased length of lashes is consistent with the ability of latanoprost to prolong the anagen phase of the hair cycle. Correlation with laboratory studies suggests that initiation and completion of latanoprost hair growth effects occur very early in anagen and the likely target is the dermal papilla.

In conclusion, Losartan, Candesartan and Telmisartan are partial agonists of the B2R through direct binding and activation, suggesting that B2R agonism could partly explain the beneficial effects of these drugs.

Until the discovery made by inventors of the present invention, there had been no reported effect of angiotensin II receptor type 1 (AT1) antagonists on secretion of prostaglandins and subtypes thereof.

As used herein, the term “subject” means an animal, preferably a mammal, and most preferably a human. A subject may be a patient having an undesired hair loss problem or a need for hair growth as discussed herein.

As used herein, the term “active Ingredient” refers to an active substance that has activity or effect of increasing/promoting hair growth and/or decreasing/delaying hair loss. The term “active ingredient” of the present invention includes a physiologically acceptable salt form of the active ingredient. The term also includes a prodrug which releases an active substance in vivo when such prodrug is administered to a subject. The term is used interchangeably with the term “drug” according to some embodiments of the present invention.

As used herein, the term “physiologically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

The term “increasing” or “promoting” refers to a positive change in the amount of hair growth. The positive change may be about 5%, about 10%, about 25%, about 50%, about 75%, about 90%, about 100% or more when compared to a control amount hair growth. For example, a composition of the present invention may increase or promote hair growth in a subject by about 5%, about 10%, about 25%, about 50%, about 75%, about 90%, about 100% or more as compared to the amount of hair growth without the application of the composition of the present invention.

As used herein, the term “reducing” or “delaying” refers to a negative change in the amount of hair loss or progression of hair loss. The negative change may be about 5%, about 10%, about 25%, about 50%, about 75%, about 90%, about 100% when compared to a control amount or progression of hair loss. For example, a composition of the present invention may reduce hair loss in a subject by about 5%, about 10%, about 25%, about 50%, about 75%, about 90%, about 100% as compared to the amount of hair loss without the application of the composition of the present invention.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 30%, preferably 20%, more preferably 10%.

As used herein, the term “comprises” means “includes, but is not limited to.”

The term “physiologically acceptable carrier” is used herein to refer to a material that is compatible with a recipient subject, preferably a mammal, more preferably a human, and is suitable for delivering an active ingredient to the target site without terminating the activity of the active ingredient. The toxicity or adverse effects, if any, associated with the carrier preferably are commensurate with a reasonable risk/benefit ratio for the intended use of the active agent.

The term “carrier” is used interchangeably herein, and Includes any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating physiologically acceptable compositions and known techniques for the preparation thereof.

The term “transdermal permeation enhancer” means a natural or synthetic molecule which facilitates the absorption of a given active agent or combination of active agents through the skin, e.g., as a portal for the administration of drugs by topical application.

A transdermal permeation enhancer may result in an increased aggregate transdermal delivery over a period of time (e.g., 3, 6, 9, 12 or 24 hours) by 5%, 10%, 25%, 50%, 100%, 2-folds, 5-folds, or more. Transdermal permeation enhancers of the present invention include, but are not limited to, those with diverse mechanisms of action including those which have the function of improving the solubility and diffusibility of the drug within the multiple polymer and those which improve percutaneous absorption, for example, by changing the ability of the skin to retain moisture, softening the skin, improving the skin's permeability, acting as permeation assistants or hair-follicle openers or changing the state of the skin including the boundary layer.

Permeation enhancers suitable for use with the present invention include, but are by no means limited to, a natural or synthetic molecules which facilitate the absorption of an active agent through skin. Some examples of permeation enhancers are polyhydric alcohols such as dipropylene glycol, propylene glycol and polyethylene glycol which enhance drug solubility; oils such as olive oil, squalene, and lanolin; fatty ethers such as cetyl ether and oleyl ether; fatty acid esters such as isopropyl myristate which enhance drug diffusibility; urea and urea derivatives such as allantoin which affect the ability of keratin to retain moisture; polar solvents such as dimethyldecylphosphoxide, methyloctyl-sulfoxide, dimethyllaurylamide, dodecylpyrrolidone, isosorbitol, dimethyl-acetonide, dimethylsulfoxide, decylmethyl-sulfoxide, and dimethylformamide which affect keratin permeability; salicylic acid which softens the keratin; amino acids which are permeation assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, isopropyl palmitate and glyceryl monooleate. The concentration of each enhancer may be between 0.1 to 20%.

In some embodiments of the present invention, the permeation enhancer is propylene glycol or azone or a combination of both.

As with percentages disclosed herein, these percentages may be mass per mass (i.e., by weight). In some embodiments, the ingredients of the topical compositions disclosed herein are provided in amounts ranging from about 1 μg to about 1 g, or higher (e.g., 1 μg-2 μg, 2 μg-5 μg, 5 μg-10 μg, 10 μg-25 μg, 25 μg-100 μg, 100 μg-500 μg, 500 μg-1 mg, 1 mg-5 mg, 5 mg-10 mg, 10 mg-20 mg, 20 mg-30 mg, 30 mg-40 mg, 40 mg-50 mg, 50 mg-60 mg, 60 mg-70 mg, 70 mg-80 mg, 80 mg-90 mg, 90 mg-100 mg, 100 mg-250 mg, 250 mg-500 mg, 500 mg-1 g, or higher, and overlapping ranges thereof). These amounts may be the weight of the ingredient per individual application (or dose), per unit or per container (tube, bottle, jar, etc.). Individual applications may be made hourly, 1-10 times or 2-5 times per day, weekly, or as needed. The concentration of the active ingredient may be from about 1 to about 100 mg per ml. e.g., from about 3 to about 50 mg per ml.

The active ingredient described herein may be formulated for administration with a pharmaceutical carrier in accordance with known techniques. See, e.g., Remington, The Science And Practice of Pharmacy (9^{th} Ed. 1995), herein incorporated by reference. In the manufacture of a composition according to several embodiments of the invention, the active ingredient (including the physiologically acceptable salts thereof) may be admixed with, inter alia, an acceptable carrier. One or more active ingredients may be incorporated in the compositions of the invention, which may be prepared by any of the well-known techniques of pharmacy comprising admixing the components, optionally including one or more accessory ingredients. Carriers which may be used include petroleum jelly, lanoline, polyethylene glycols, alcohols, transdermal enhancers, and combinations of two or more thereof. In some embodiments, the topical composition is partially or fully incorporated in delivery vehicles such as microspheres or nanoparticles, or is encapsulated (e.g., in liposomes). In some embodiments, a topical composition is pre-impregnated in/on a support structure (e.g., tape, patch, bandage, etc.).

The compositions of the present invention may comprise one or more active ingredients according to some embodiments. In addition, the compositions disclosed herein comprise other additives, such as pH-adjusting additives. In some embodiments, useful pH-adjusting agents include acids, such as citric acid or lactic acid, bases or buffers, such as sodium lactate, sodium acetate, sodium phosphate, sodium citrate, sodium borate, or sodium gluconate.

The compositions may contain antimicrobial preservatives in some embodiments. In several embodiments, antimicrobial preservatives include, but are not limited to, methylparaben, propylparaben, benzyl alcohol, ethylhexylglycerin, potassium sorbate, phenoxyethanol, EDTA, grapefruit seed extract, tea tree oil, sodium benzoate, dehydroacetic acid, and combinations thereof. In some embodiments, anti-fungal preservatives are used alone or in combination with anti-bacterial preservatives.

### EXAMPLES

The present description is further illustrated by the following examples, which should not be construed as limiting in any way. The contents of all cited references (including literature references, issued patents, published patent applications as cited throughout this application) are hereby expressly incorporated by reference.

### Example 1

We studied angiotensin II receptor antagonists activating bradykinin (BK) B2 receptors to promote secretion of Prostaglandin. Losartan, candesartan, telmisartan were selected for the study described below, and bradykinin was used as control. Implantation Two cell lines were transferred into T-75 cell culture flask, and then the instructions of PG kit were strictly followed when cells have reached the appropriate density. Day 1 Cells were seeded in 24 well dishes with minimum medium and allowed to adhere overnight. Day 2 Cells were starved in the serum-free conditioned medium. Day 3 Minimum medium were used, and Losartan, Candesartan, Telmisartan were added to the medium respectively, the amount was 10 μM; the cells were incubated at 37° C. for 1 h. The supernatant was collected after incubation for later use. Day 4 24 well dishes were washed and then dyed for 45-90 min, and then the OD value was measured for analysis. Therefore OD values of BK B2 receptor agonists were obtained and the EC50, EMAX were calculated. The cell strains used in the study were human primary vein endothelial cells (HPVEC) and human aortic smooth muscle cells (HASMC).

### Example 2

In order to detect endogenous prostaglandin, enzyme immunoassay (EIA) was used to determine the amount of prostaglandin and subtypes thereof in the cell supernatant described above. The EIA protocol took 18 hours, and the results were shown in Table 1 below. The results indicated the effect of 10 μM of Candesartan was roughly equal to 50% of the effect of 1 μM of bradykinin, while the effect of 30 μM of Candesartan was nearly equal to 90% of the effect of 1 μM of bradykinin. Moreover, the effect of 30 μM of Losartan was nearly equal to 60% of the effect of 1 μM of bradykinin; and the effect of 30 μM of Telmisartan was nearly equal to 25% of the effect of 1 μM of bradykinin. Therefore, the order of decreasing efficiency of the tested Angiotensin II receptor antagonists is: Candesartan, Losartan, and Telmisartan.

TABLE 1
The Relative Amount of PG Secretion
PG secretion amount caused by 1 μM bradykinin was set as 100%

| | PG release (%) compared with BK-1 μM | | |
|---|---|---|---|
| | (100%) | | |
| Concentration | Candesartan | Telmisartan | Losartan |
| 30 μM | 86.83 ± 7.21 | 25.46 ± 3.48 | 58.17 ± 6.32 |
| 10 μM | 46.19 ± 5.65 | 14.56 ± 2.42 | 23.18 ± 3.65 |
| 3 μM | 26.91 ± 4.13 | 13.56 ± 1.56 | 12.06 ± 4.12 |
| 1 μM | 16.51 ± 2.02 | 9.01 ± 1.35 | 8.16 ± 2.43 |
| 300 nm | 12.91 ± 3.08 | 6.46 ± 3.1 | 4.69 ± 1.21 |
| 100 nm | 11.27 ± 2.74 | 4.69 ± 1.78 | 2.96 ± 2.22 |
| 30 nm | 6.21 ± 2.3 | 4.48 ± 1.54 | 0.18 ± 0.74 |

The detailed protocol for the elucidation of inhibited PG secretion by B2 receptor inhibitor HOE-140 was shown as follows. Two different cell lines were transferred into T-75 cell culture flask, and then the instructions of PG kit were strictly followed when cells have reached the appropriate density. Day 1. Cells were seeded in 24 well dishes with minimum medium and allowed to adhere overnight. Day 2. Cells were starved in the serum-free conditioned medium. Day 3. Minimum medium were used, and 10 μM of Losartan, Candesartan, and Telmisartan were added to the medium, respectively, and 1 μM of HOE-140 was added at the same time. Cells were incubated at 37° C. for 1 h. The supernatant was collected after incubation for later use. Day 4. 24 well dishes were washed and then dyed for 45-90 min, and then the quantitative analysis was carried out. Therefore OD values (420 nm) of BK B2 receptor agonists were obtained and the EC50, EMAX were calculated.

The results of quantitative study shown below in Table 2 indicate that 1 μM HOE-140 could block the 85% of PG synthesis caused by 10 μM of Candesartan, or 80% of PG synthesis caused by 10 μM of Losartan or 10 μM of Telmisartan. Therefore we could conclude the angiotensin II type 1 receptor antagonists influence PG secretion via B2 receptor since HOE-140 acts as B2 receptor antagonist

TABLE 2
Relative PG Secretion Amount

PG relaease (%)
compared with BK-1 μM (100%)

| | 10 μM | 10 μM + 1 μM HOE-140 |
|---|---|---|
| Candesartan | 46.19 ± 5.65 | 6 ± 3.12 |
| Telmisartan | 14.56 ± 2.42 | 3 ± 2.03 |
| Losartan | 23.18 ± 3.65 | 5 ± 4.45 |

### Example 3

In order to study of prostaglandin subtypes secretion triggered by activation of B2 receptor by angiotensin II Type 1 receptor antagonists, candesartan was used here as angiotensin II type 1 receptor antagonist to induce human primary vein endothelial cells (HPVEC) and human aortic smooth muscle cells (HASMC) PG secretion, following the same protocol above; the amount of Candesartan added was 10 μM. Then EIA was employed to determine the amount of PG subtypes, and the results shown below in Table 3 indicated that 30% of PG secretion induced by 10 μM of Candesartan was Prostaglandin E2, while roughly 6% was Prostaglandin F-2a and there was no Prostaglandin D detected (table 3). These percentage values above were all calibrated with respect to bradykinin. However bradykinin failed to penetrate through skin, and moreover, it has obvious adverse effect and toxicity, hence bradykinin has yet become clinical medication.

TABLE 3
The PG Subtype Percentage

| Cell Strain | PGE2 (%) | PGF-2a (%) |
|---|---|---|
| HASMC | 37.13 ± 9.85 | 6.21 ± 2.56 |
| HPVEC | 32.54 ± 6.93 | 5.47 ± 3.32 |

HPVEC: Human Primary Vein Endothelial Cells;
HASMC: Human Aortic Smooth Muscle Cells

### Example 4

B2 receptor binding was analyzed by homology modeling techniques. AT1R and the B2R both belong to the superfamily of seven transmembrane (7TM) or G protein-coupled receptors (GPCRs), share sequence identity of about 30% and most likely bind small molecule ligands in comparable binding pockets in the transmembrane domains [1]. Accordingly, we want to carry out a study of the interaction between Losartan (which is an AT1R agonist) and B2R. Although computational studies have previously been performed to investigate hormone/ligand-GPCR complex structures at the molecular level, only two crystal GPCR structures have been solved: the ground state rhodopsin (Rh) structure and the inactive form of the β2-adrenergic receptor (β2-ADR). Bradykinin receptors B1 and B2 belong to the class A family of the rhodopsin-like GPCRs. Since the B2R structure is not available, the high-resolution crystal structure of bovine rhodopsin (PDBID: 1HZX) was used as a template for the B2 receptor modeling by using homology modeling techniques. The B2 receptor sequence (entry P30411) was taken from the Swiss-Prot Data Base. An initial 3D homology model was built by using the MACROMOLECULES module from the Discovery Studio 3.5 software package. MD simulation is performed by using CHARMm forcefield. Then the model structure was further evaluated using The Ramachandran Plot and the Verify Protein (Profiles-3D) protocol (Table 4).

Structures of Losartan, Candesartan, and Telmisartan were obtained from Chemdraw 3.0 and CHARMm forcefield was applied. Building a model for the Losartan-B2R complex ligand docking: The receptor pocket is conserved within the class A GPCR family. The Input Site Sphere of the receptor pocket is located −0.164482, 6.6825, 22.61, 22.4 respectively. We launch the CDOCKER which is a powerful CHARMm-based docking method to generate highly accurate docked poses. A random conformation of Losartan is docked into the BK B2 receptor binding site (Table 5).

Residues found at the Losartan binding pocket border are S87, S88, C89, T90, A92, E93, TM2, T264, E265, R267, TM6 of A268, Y347, R338, F339, V350 of the C-term. Similarly, residues found at the Candesartan binding pocket border are S87, S88, C89, T90, A92, E93, TM2, D154, R155, A158, TM3 of R167, T264, E265, R267, A268, L271, TM6 of V272, Y347, R338 of C-term-loop. Therefore we can conclude that these two drugs were binding into the same pocket (Table 6).

TABLE 4
Pharmacophore Features of Three AT1B

| Candesartan | Losartan | Telmisartan |
|---|---|---|
| 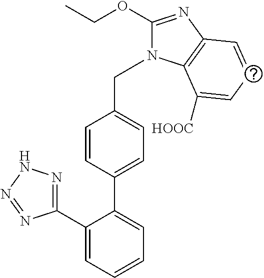 [SMILES: CCOc1nc2cccc(C(=O)O)c2n1Cc1ccc(-c2ccccc2-c2nn[nH]n2)cc1] | 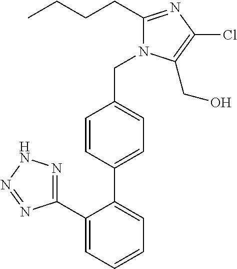 [SMILES: CCCCc1nc(Cl)c(CO)n1Cc1ccc(-c2ccccc2-c2nn[nH]n2)cc1] | 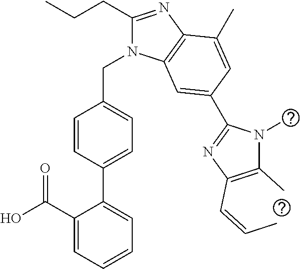 [SMILES: C/C=C\c1nc(-c2cc(C)c3nc(CCC)n(Cc4ccc(-c5ccccc5C(=O)O)cc4)c3c2)n(C)c1C] |
| 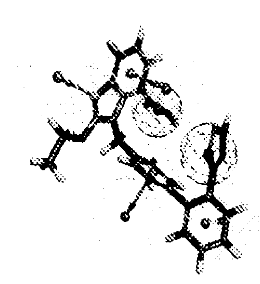 | 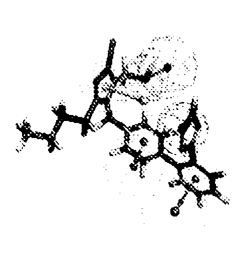 | 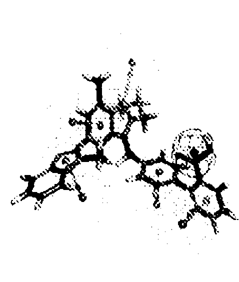 |

Usually the pharmacophore features also include hydrophobicity (Light Blue), aromaticity (Yellow), hydrogen bond acceptor (Green), hydrogen bond donor (Violet), negative ionization (Dark Blue).
Each HBA function has two features because of the directionality of hydrogen bond: the position of related heavy atoms and the projection spot, which is the hydrogen bond direction toward the target.
Each sphere defines the spatial tolerance of a specific site.

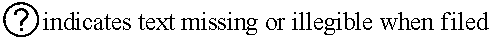

TABLE 5
Intracellular view of the activated B2R

| Candesartan | Losartan |
|---|---|
| 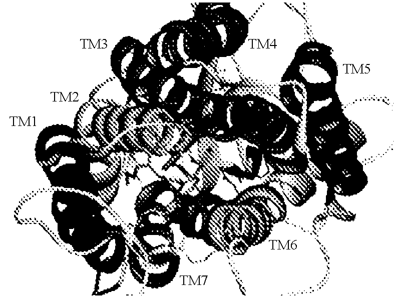 | 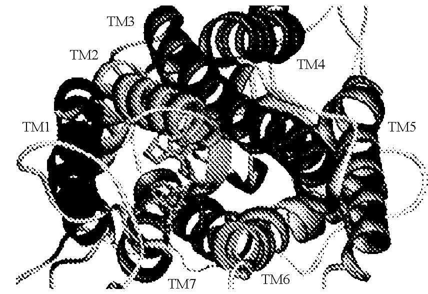 |

According to Table 2, the color of the helices are: TM1: Blue; TM2, green; TM3: cyan; TM4: orange; TM5: violet; TM6: yellow; TM7: magenta.

TABLE 6
Interaction between Ligands and Residues

| Candesartan | Losartan |
|---|---|
| 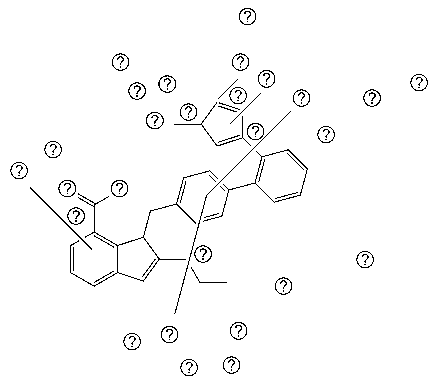 | 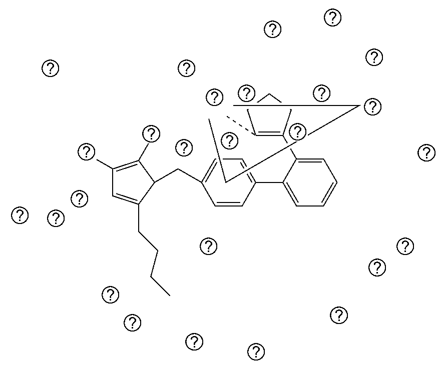 |
| 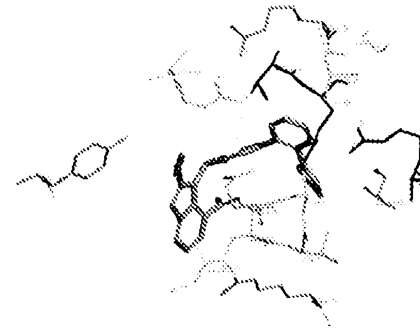 | 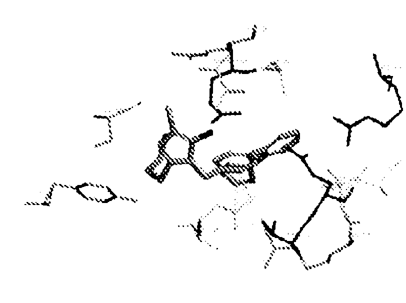 |

In the 2D map above, green arrow stands for hydrogen bond.
The green dotted line represents the interaction with hydrogen bond of amino acid main chain, the arrow points to the electron donor.
Meanwhile the blue dotted arrow represents the interaction with amino acid side chain and points towards electron donor.
Green circles represent the residues interacted via Van Der Waals force.
Blue hale around residues stands for solvent-accessible interaction, and the radius is proportional to the solvent-accessible surface.
The hydrogen bond between ligands and acceptors are represented with green dotted line.
The hydrogen atoms are omitted in the map, PI electron interaction is represented with an orange line, and colors differ as amino acids vary.

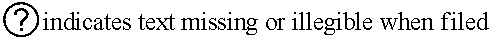

In the case of Candesartan (FIG. 2), the R155 and R167 of TM3 interact with the E265 and R267 and the ring of R338. The 3 nitrogen atoms of tetrazole ring form hydrogen bond with R155 (2H bond) and E265, the distances are: 2.9 Å, 3.0 Å, and 3.2 Å, respectively. Also, there's PI interaction between the ring and R155; biphenyl and phenyl group residues have PI interaction with R155, R167, R267 and R338. VDW interaction and solvent effect with residues nearby are shown in the Table 6.

Losartan could interact with the ring of R267 and R338 of TM6. The tetrazole and the biphenyl residue form strong PI-electron interactions between R267 of TM6 and R338 of C-term. Two nitrogen atoms of the tetrazole ring of losartan may also form hydrogen bonds with R267 and R338, with the distance of 3.2 Å, 3.1 Å respectively. Moreover, Losartan shows VDW interaction and solvent effect with residues nearby, which are represented in the table 4,5 and 6. We could thus conclude losartan has no interaction with TM3, while candesartan interacts with R155 and R167 of TM3 via Hydrogen bond and PI electron interaction, which makes it bind BK B2 more efficiently (Shown in Table 7).

TABLE 7
Part of the Ribbon Structures in Ligand Binding Site

| Candesartan | Losartan |
|---|---|
| 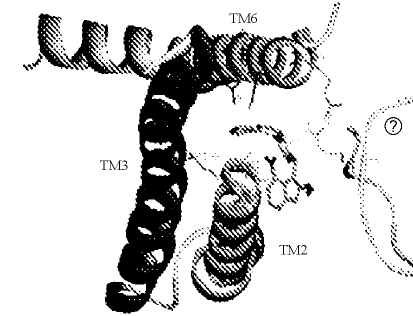 | 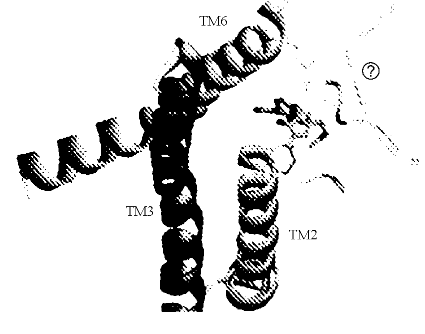 |
| 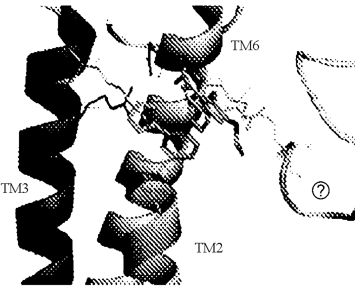 | 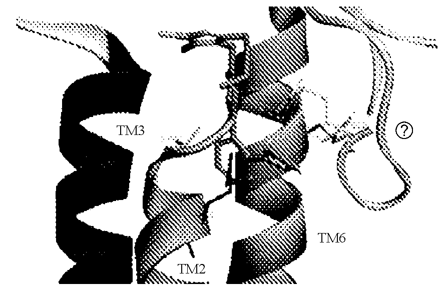 |

The helices are colored as: TM2: green; TM3: brown; TM6: yellow.
The frame structures of Losartan and Candesartan are colored based on the elements.
The key residues are represented by a code consisting three letters.
Green dotted lines represent the hydrogen bond between ligands and acceptors.
The H atoms are omitted.
The orange lines represent PI interaction.
Different amino acids are colored in differently.

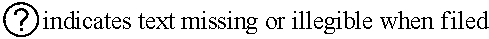

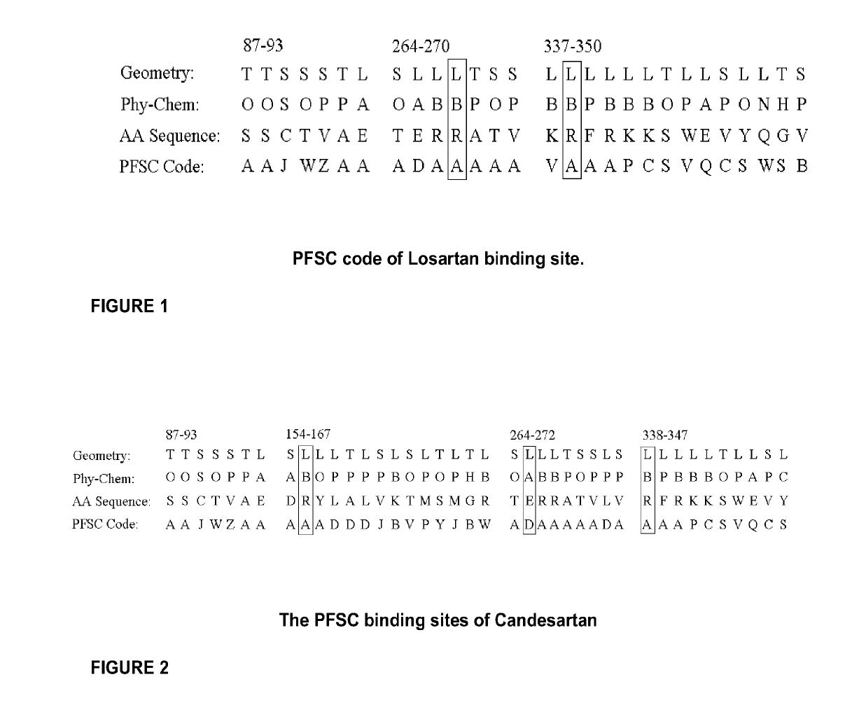

Meanwhile, we use PFSC code to describe the three-dimensional protein binding site. The numbers represent binding sites on the chain. Blue frames represent hydrogen bond and PI interaction which amino acids involve. The results are shown in FIG. 1 and FIG. 2. Geometry: the amino acid properties illustration, tiny=T, small=S, large=L hardiness=H. Phy-Chem=Chemical-Physical Properties, hydrophobicity=P, acidic=A, basic=B, the Glycine=H, OH═O, carbonyl and amide group O═CNH2=N, SH═S. PFSC Code: Structural assignments from PFSC method, “A” indicating α-helix and “B” β-strand. The PFSC in violet represent secondary structure like carriers.

According to the number of binding sites, the binding affinity in descending order is: Candesartan, Losartan, and Telmisartan. Candesartan has stronger binding affinity than Losartan according to the number of binding sites (Table 8 and 9); meanwhile Telmisartan is the least likely to bind acceptors. Therefore the difference between functional groups determines the affinity of the drugs, which is the molecular basis of our patent claims.

TABLE 8
Number of Pharmacophore Features of AT1 Blockers

| | | | hydrogen | hydrogen | |
|---|---|---|---|---|---|
| | negative | | bond | bond | hydro- |
| | ionizable | aromatic | acceptor | donor | phobic |
| losartan | 1 | 3 | 0 | 1 | 0 |
| candesartan | 2 | 3 | 1 | 0 | 1 |
| telmisartan | 1 | 4 | 1 | 0 | 0 |

TABLE 9
Residues Involved in Interactions

| | hydrogen bond | hydrogen | |
|---|---|---|---|
| | acceptor | bond donor | PI interaction |
| losartan | R267, R338 | 0 | R267, R338 |
| candesartan | R155 | E265 | R155, R167, |
| | | | R267, R338 |

### Example 5

To explore the optimized dosage form and preparation technology of AT1R antagonists, we selected Candesartan, which has the long-term stability for preservation, as a sample from three AT1R antagonists (Losartan, Candesartan, Telmisartan) and prepared three kinds of dosage form, analyzed by transdermal experiments and high-performance liquid phase (HPLC) content analysis. Three dosage forms (liniments, gels and ointments) of Candesartan were prepared in different concentrations (0.03%, 0.3% and 3%). Then, different proportional of transdermal promoters was added to perform transdermal experiments. The transdermal effect of three kinds of Candesartan formulation was evaluated by the modified Franz diffusion pool. Cumulative transmittance of liniments, gels and ointments in 0.3% concentration of Candesartan for 9 hours was measured respectively. Results: (1) The best transdermal promoter ratio of liniments, gels and ointments were 2% propylene glycol+2% azone, 4% propylene glycol, and 5% propylene glycol+2% azone. (2) 9 hours of cumulative transmittance of 0.03%, 0.3%, 3% liniments were 3.813%, 9.592% and 10.963%, respectively; 9 hours of cumulative transmittance of 0.03%, 0.3%, 3% gels were 1.092%, 3.299% and 10.705%, respectively; 9 hours of cumulative transmittance of 0.03%, 0.3%, 3% ointments were 1.808%, 0.182% and 0.038%, respectively. Conclusion: The different contents of Candesartan liniments, gels and ointments are all have some transdermal abilities, and the abilities are: liniments>gels>ointments. The AT1R antagonists described herein, such as Losartan, Candesartan, Telmisartan, are innovative dual-target drug for hair growth promotion.

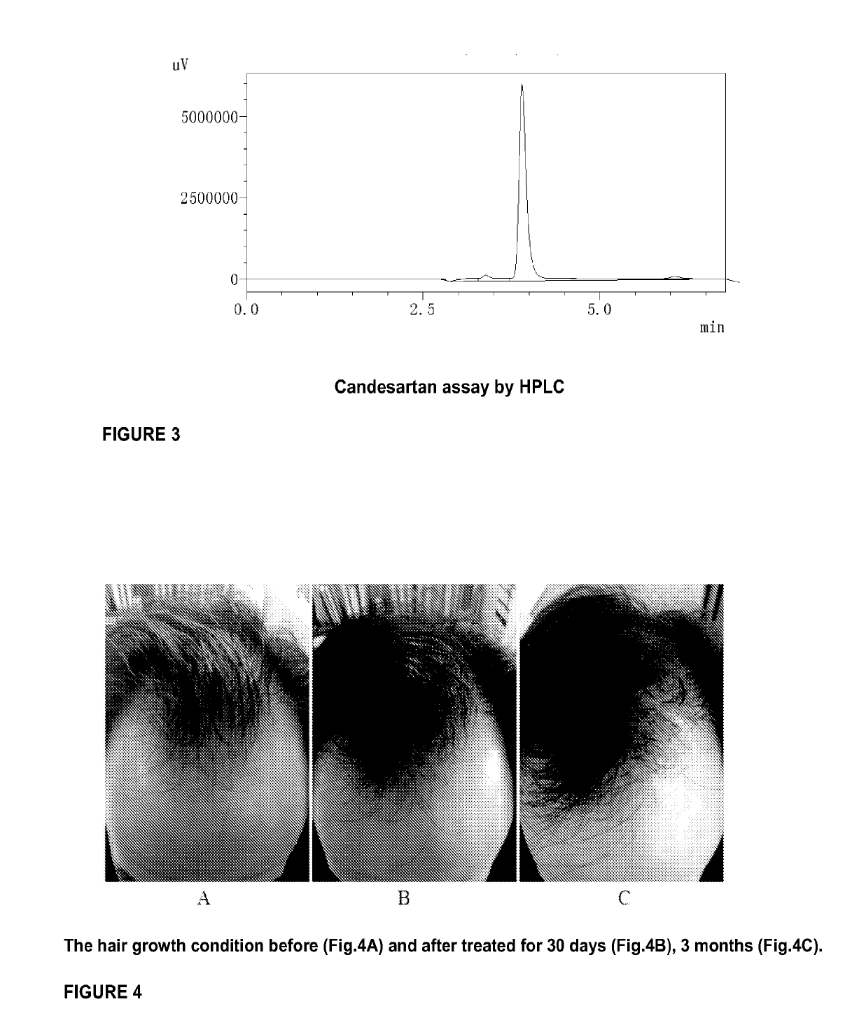

To determine the optimal dosage form of Candesartan and best transdermal promoter, we plan to use transdermal test combined with HPLC content analysis as screening basis, research on dosage forms and transdermal promoter to achieve a stable and reliable preparation. The samples were analyzed by HPLC on an Agilent TC-C18 column (150 mm×4.6 mm, 5 microns). The mobile phases were methanol and 0.02 mol/L potassium dihydrogen phosphate buffer (66:34, pH was adjusted to 4 with phosphoric acid). Flow rate: 1 ml/min; Column temperature: 30° C. Excitation wavelength and fluorescent wavelength of fluorescence detection were 267 nm and 380 nm, respectively. Under this condition, Candesartan retention time was 3.898 min, target compounds peak shape is good which could be effectively separated, and no impurity peak, baseline is smooth (shown as FIG. 3).

### Example 6

In order to optimize the candesartan formulation, the liniments, gels and ointments three dosage forms of Candesartan were prepared, and in each dosage form there were three concentrations, which were 0.03%, 0.3%, 3%, respectively. Then, the best dosage form and concentration were determined. In order to prepare liniment for candesartan, we mixed candesartan, ethyl alcohol, 0.1M NaOH, propylene glycol, azone and glycerol and added distilled water to full amount, and stirred to dissolve.

In order to prepare Candesartan solid dispersions, we weighed Candesartan and PVPK-30, with the right amount of mixed solvent (methylene chloride, methanol=1:1) heating and stirring to dissolve Candesartan, then ethanol dissolves the carrier, to be fully dissolved to clarify. Two kinds of solution was mixed and stirred to clear solution. Then, it was put into the round bottom flask, 45° C. reduced pressure distillation to dry solvents. After removing solvent to become white solid, then, it was rapidly frozen −20° C. for 2 h, and then placed in 60° C. oven to remove residual solvent, inside the dryer saved for later use.

Candesartan gels were obtained as follows. Propylene glycol, azone, glycerin, caprylyl glycol, ethylparaben were mixed and heated to dissolve; Candesartan was added, with full amount of water, and stirred to dissolve; carbomer 940 was added for overnight swelling, then appropriate amount of tiethanolamine was added to form transparent gels.

0.03% (by Weight) Candesartan:

Glycerylmonostearate, stearic acid, white petrolatum, and liquid paraffin is heated to melt as oil phase. Glycerol, SDS, ethyl paraben and water were heated to 90° C. as aqueous phase. Candesartan was added into propylene glycol, heated until completely dissolved. The oil phase was then slowly added into the aqueous phase, and Candesartan was added with stirring. Stirring was continued until the temperature lowered to room temperature. Then, Azone was added. Keep stirring until the solution condensate.

0.3% (by Weight) Candesartan:

Glycerylmonostearate, stearic acid, white petrolatum, and liquid paraffin is heated to melt as oil phase. Glycerol, SDS, ethyl paraben and water were heated to 90° C. as aqueous phase. Candesartan was added into propylene glycol and PEG400, and heated until completely dissolved. The oil phase was then slowly added to the aqueous phase, and Candesartan was added with stirring. Stirring was continued until the temperature lowered to room temperature. Then, Azone was added. Keep stirring until the solution condensate.

3% (by Weight) Candesartan:

Candesartan was comminuted through 60 mesh sieve. Glycerylmonostearate, stearic acid, white petrolatum, and liquid paraffin were heated to melt as oil phase. Glycerol, SDS, ethyl paraben, propylene glycol and water were heated to 90° C. as aqueous phase. The oil phase was then slowly added to the aqueous phase. After continuous stirring to dissolve and cooling, Azone was added. Keep stirring until the solution condensate. The Candesartan was added to the matrix with stirring evenly.

### Example 7

To study transdermal effect of different prescription compatibility, the 0.3% content of the liniments, gels and ointments samples were selected to be used in the first round of screening, and the results were shown in table10; After the first round of screening, the optimal concentration of transdermal promoter and compatibility was determined, and on this condition, the other 2 concentration (0.03%, 3%) were compared.

TABLE 10
The results of the first round of screening of transdermal absorption
promoting agent

transdermal absorption
promoting agent (%)

| Dosage form | No. | propylene glycol | Azone |
|---|---|---|---|
| Liniments | 1 | 0 | 0 |
| | 2 | 4 | 0 |
| | 3 | 0 | 4 |
| | 4 | 2 | 2 |
| Gels | 1 | 0 | 0 |
| | 2 | 2 | 0 |
| | 3 | 4 | 0 |
| Ointment | 1 | 5 | 0 |
| | 2 | 5 | 2 |
| | 3 | 5 | 4 |

The abdominal hair of mice was removed. The mice were sacrificed by spinal dislocation. The subcutaneous fat was removed, and the depilous skin of abdomen was reserved in 4° C. physiological saline. Skin was fixed between the supply room and reception room facing the supply room. The effective permeability area was 4 cm². Receiving liquid was physiological saline, and receiving chamber volume V were 25 ml. 1.0 g of the tested sample was coated evenly on the effective skin surface with the electromagnetic stirrer run at a speed of 300 r/min. To take out the receiving liquid in 1, 2, 3, 4, 5, 6, 7, 8, 9 h, 0.5 ml/h (Complementing the samequantity each time), the samples were centrifuged and the supernatant was took for HPLC analysis.

After the detection, the cumulative transmittance P (%) was calculated:

$$P ( \% ) = \frac{Cn \times V + {\sum }_{i = 1}^{n - 1} Ci \times 0.5}{m} \times 100 \%$$

V: the volume of receiving liquid in receiving room; Ci: drug concentration in receiving liquid between the time to last time of the i time; Cn: drug concentration in the receiving liquid when the n time taking the sample; M: drug content in the sample.

To detect the content of Candesartan of receiving liquid by HPLC, the optimal dosage forms and the best proportion of transdermal absorption promoting agent were selected. 1.0 g/L Candesartan standard stock solution was prepared as follows. 10.0 mg of Candesartan was added into methanol solution, once melt it and diluted to 10 ml scale, and mixed well. The concentrations of 80 mg/L, 20 mg/L, 10 mg/L, 5 mg/L, 1 mg/L of Candesartan methanol standard solution were prepared from standard stock solution.

The Standard Curve was Obtained as Follows.

950 μl of normal saline, was added into 50 μl series of different concentration of standard solution to acquire the standard samples which included 4000, 1000, 500, 250, 50 μg/l Candesartan. 20 μl each time was injected, and the map recorded, the concentration (μg/l) was as the abscissa, peak area was as the vertical coordinates, regression equation was Y=12224x+437621, r²=0.9998, and the linear range was 50 μg/l˜4000 μg/l.

According to the operation of the standard curve method, high, medium and low three concentrations (1000, 500, 250 μg/l) of the standard samples were prepared. The results of the recovery rate were shown in table 11.

TABLE 11
The recovery rate

| | Addition | Measured | Recovery | Average | |
|---|---|---|---|---|---|
| No. | μg/l | Amount/μg/l | Rate/% | recovery rate/% | RSD % |
| 1 | 250 | 280.584 | 112.2335 | 99.9453 | 8.042 |
| 2 | 250 | 273.278 | 109.3111 | | |
| 3 | 250 | 238.020 | 95.2079 | | |
| 4 | 500 | 505.056 | 101.0112 | | |
| 5 | 500 | 424.418 | 84.8836 | | |
| 6 | 500 | 476.436 | 95.2871 | | |
| 7 | 1000 | 1004.931 | 100.4931 | | |
| 8 | 1000 | 989.696 | 98.9696 | | |
| 9 | 1000 | 1021.103 | 102.1103 | | |

Three different dosage forms of the 0.3% Candesartan were used as screened samples firstly to explore the optimal contents and compatibilities of the permeation enhancers. The results indicated that 2% propyleneglycol+2% azone was best for the transdermal effect of the liniments, 4% propyleneglycol was best for the gels, and 5% propylene glycol+2% azone was best for the ointments (Table 12, Table 13).

TABLE 12
Screening of the Permeation Enhancers

| | | | Cumulative | |
|---|---|---|---|---|
| | | | Penetration | |
| | Content of the Penetration | | Rate | Drug Release |
| Dosage | Enhancers (%) | | of 9 Hours | Fitting |
| Forms | propyleneglycol | azone | (%) | Equation |
| 0.3% | 0 | 0 | 6.549 | First-order |
| liniments | 4 | 0 | 0.521 | Zero-order |
| | 0 | 4 | 4.584 | Higuchi |
| | 2 | 2 | 9.592 | Zero-order |
| 0.3% | 0 | 0 | 0.273 | First-order |
| gels | 2 | 0 | 0.941 | Zero-order |
| | 4 | 0 | 3.299 | Higuchi |
| 0.3% | 5 | 0 | 0.142 | Zero-order |
| ointments | 5 | 2 | 0.182 | First-order |
| | 5 | 4 | 0.154 | First-order |

TABLE 13
The Optimal Contents and Compatibilities of the Permeation Enhancers

Content of the
Penetration Enhancers (%)

| Dosage Forms | propylene glycol | azone |
|---|---|---|
| liniments | 2 | 2 |
| gels | 4 | 0 |
| ointments | 5 | 2 |

According to the results of screening of the permeation enhancers, liniments, gels and ointments which contained 0.03%, 0.3% and 3% Candesartan respectively were detected the cumulative permeation rate for 9 hours. The results showed that the transdermal effects were liniments>gels>ointments. The middle and high concentrations of liniments could attain good transdermal effects, and high concentration of gels indicated high permeation rate. However, three concentrations of ointments didn't show good transdermal effects. In addition, the permeation rate declined with the content of Candesartan increasing, which implied PEG400 may hinder the transdemal absorption and the form of Candesartan dispersing in the cream base may affect the transdemal effect (Table 14).

TABLE 14
Transdermal effects of 3 dosage forms of candesartan

| | | | | Cumulative |
|---|---|---|---|---|
| | Content of | Penetration Enhancers | | Penetration |
| Dosage | Candesartan | (%) | | Rate of 9 Hours |
| Forms | (%) | propylene glycol | azone | (%) |
| liniments | 0.03 | 2 | 2 | 3.813 |
| | 0.3 | 2 | 2 | 9.592 |
| | 3 | 2 | 2 | 10.963 |
| gels | 0.03 | 4 | 0 | 1.092 |
| | 0.3 | 4 | 0 | 3.299 |
| | 3 | 4 | 0 | 10.705 |
| ointments | 0.03 | 5 | 2 | 1.808 |
| | 0.3 | 5 | 2 | 0.182 |
| | 3 | 5 | 2 | 0.038 |

### Example 8

The angiotensin II type 1 receptor antagonist suspension with 0.3% concentration was obtained for clinical use as described above. The suspension was applied to young and middle-aged patients (cover the areas where hair loss appeared) suffering from androgenetic alopecia, or seborrheic alopecia twice a day, for 3 months; hair growth was observed. The treated areas were observed daily to monitor hair growth and side effects, e.g. swelling, pruritus, pain, etc. And the treated parts were photographed weekly. As shown in FIG. 4, the photos suggested that obvious improvement appeared after a week. And it could be seen that tiny hairs grew out in the parts used to be bald, as shown in FIG. 4(B). The even more obvious result could was shown in FIG. 4(C).

### REFERENCES

1. Calixto J B, Cabrini D A, Ferreira J, Campos M M. Kinins in pain and inflammation. Pain 2000; 87:1-5.
2. Heitsch H. Bradykinin B2 receptor as a potential therapeutic target. Drug News Perspect 2000; 13:213-225.
3. Sasaki S, Hozumi Y, Kondo S. Influence of prostaglandin F2a and its analogues on hair regrowth and follicular melanogenesis in a murine model. Exp Dermatol. 2005; 14:323-328.
4. Alhenc-Gelas F, Bouby N, Richer C, Potier L, Roussel R, Marre M. Kinins as therapeutic agents in cardiovascular and renal diseases. Curr Pharm Des. 2011; 17(25):2654-2662.
5. Johnstone M A, Albert D M. Prostaglandin-induced hair growth. Surv Ophthalmol. 2002; 47 Suppl 1:S185-202.
6. Bonde M M, Olsen K B, Erikstrup N, Speerschneider T, Lyngsø C, Haunsø S, Nielsen M S, Sheikh S P, Hansen J L. The angiotensin II type 1 receptor antagonist Losartan binds and activates bradykinin B2 receptor signaling. Regul Pept. 2011; 167(1):21-25.
7. Gether U, Kobilka B K. G Protein-coupled Receptors. J Biol Chem, 1998; 273(29): 17979-17982.
8. Marvin C. Gershengorn and Roman Osman, Minireview: Insights into G Protein-Coupled Receptor Function Using Molecular Models. Endocrinology, 2001; 142: 2-10.
9. Ha S N, Hey P J, Ransom R W, Harrell C Jr, et al, Binding modes of dihydroquinoxalinones in a homology model of bradykinin receptor 1. Biochemical and Biophysical Research Communications. 2005; 331:159-166.
10. Su D S, Markowitz M K, DiPrado R M, Murphy K L, Harrell C M, O'Malley S S, Ransom R W, Chang R S, Ha S, Hess F J, Pettibone D J, Mason G S, Boyce S, Fredinger R M, Bock M G. Discovery of a potent, non-peptide Bradykinin B1 receptor antagonist, J. Am. Chem. Soc. 2003; 125:7516-7517.
11. Underwood D J, Strader C D, Rivero R, Structural model of antagonist and agonist binding to the angiotensin II, AT, subtype, G protein coupled receptor, Chem Biol. 1994; 1:211-221.
12. Bhuiyan M A, Ishiguro M, Hossain M, Naka mura T, Ozaki M, Miura S, Nagatomo T. Binding sites of valsartan, Candesartan and Losartan with angiotensin II receptor 1 subtype by molecular modeling. Life Sci. 2009; 85:136-140.
13. Gieldon A, Lopez J J, Glaubitz C, Schwalbe H. Theoretical Study of the Human Bradykinin—Bradykinin B2Receptor Complex. ChemBio Chem, 2008; 9:2487-2497.
14. Tand X J, Chen M. Advanced in Research of Angiotensin Receptor Blockades—Candesartan. Advances In Cardiovascular Diseases, 2004; 25(3):172-175.
15. Hu Q, Song H, Yang L, at al. The Preparation and Clinical Application of Benzene Liquid Liniment, The Chinese and Western Medical Journal, 2011; 9(6): 63-64.
16. Chen H, Wong T, Chang X, et al. Preparation of Ibuprofen Microemulsion and Its Transdermal Absorption. Chinese Pharmaceutical Journal, 2004, 39(1): 43-45.
17. Li R, Wang X, Guo Se, et al. Content Determination of Candesartan in Rats Blood Serum by HPLC. Pharmacy Today, 2011, 21(8): 484-486.
18. Du S, Gu R, Chen H. The Screening and Stability Study of the Prescription of Cream Base. Chongqing Medical Journal, 2002, 31(7): 632-633.
19. He K B, Wang Y Z, Feng A L. The Study of the Preparation of Nanoemulsion Gel of SophoraFlavescens and Lumbricus and Its Transdermal Absorption in vitro. China Journal of Chinese Materia Medica, 2013; 38(016): 2623-2627.
20. Lin G T, Sheng H G, Zhang C. Study on vitro Transdermal Absorption of FufangChansuZhengtong Cataplasm, Chinese Journal of Experimental Traditional Medical Formulae, 2011; 17(9): 1-1.
21. Wang D, Han G Z, Chen Z J. Effects of Penetration Enhancers on in vitro Percutaneous Permeation of Tea Polyphenols Liniment. Chinese Journal of New Drugs, 2010; (13): 1171-1174.
22. Ye C Q, Yu C X, Yang X H. Content Determination of Candesartan in Human Plasma by HPLC-FLU. China Pharmaceuticals, 2011; 20(17): 20-21.
23. Huang J G, Li G, Si L Q. Content Determination of Candesartan in Plasma by HPLC. China Pharmacist, 2005; 8(4): 293-294.
24. Nelson A M, Loy D E, Lawson J A, Katseff A S, Fitzgerald G A, Garza L A. Prostaglandin D2 inhibits wound-induced hair follicle neogenesis through the receptor, Gpr44. J Invest Dermatol. 2013; 133(4):881-9.
25. Garza L A, Liu Y, Yang Z, Alagesan B, Lawson J A, Norberg S M, Loy D E, Zhao T, Blatt H B, Stanton D C, Carrasco L, Ahluwalia G, Fischer S M, FitzGerald G A, Cotsarelis G. Prostaglandin D2 inhibits hair growth and is elevated in bald scalp of men with androgenetic alopecia. Sci Transl Med. 2012; 4(126):126-134.
26. Colombe L, Michelet J F, Bernard B A. Prostanoid receptors in anagen human hair follicles. Exp Dermatol. 2008; 17(1):63-72.
27. Böhm M, Schiller M, Luger T A. Non-pigmentary actions of alpha-melanocyte-stimulating hormone-lessons from the cutaneous melanocortin system. Cell Mol Biol (Noisy-le-grand). 2006; 52(2):61-8.
28. Malkinson F D, Geng L, Hanson W R. Prostaglandins protect against muine hair injury produced by ionizing radiation or doxorubicin. J Invest Dermatol. 1993; 101(1 Suppl):135S-137S.

1-41. (canceled)

42. A topical composition comprising at least one active ingredient selected from the group consisting of a bradykinin (BK) B2 receptor agonist and an angiotensin II receptor (AT1R) antagonist.

43. The topical composition according to claim 42, wherein said active ingredient is present at a concentration of from about 0.01% to about 10% by weight.

44. The topical composition according to claim 42, wherein said active ingredient is present at a concentration of from about 0.03% to about 3% by weight.

45. The topical composition according to claim 42, wherein said active ingredient is a non-peptide compound.

46. The topical composition according to claim 42, wherein said active ingredient is Losartan, or Candesartan, or Telmisartan.

47. The topical composition according to claim 42, wherein said active ingredient is 2-ethoxy-1-[[2′-(1H-tetrazol-5-yl)biphenyl-4-yl]methyl]benzlimidazole-7-carboxylic acid, 1-(cyclohexyloxycarbonyloxy)ethyl-2-ethoxy-1-[[2′-(1H-tetrazol-5-yl)biphenyl-4-yl]methyl]benzimidazole-7-carboxylate, or 2-ethoxy-1-[[2′-(2,5-dihiydro-5-oxo-1,2,4-ox-adiazol-3-yl)biphenyl-4-yl]methyl]benzimidazole-7-carboxylic acid.

48. The topical composition according to claim 42, further comprising at least a pharmaceutical carrier selected from the group consisting of transdermal permeation enhancer, transdermal absorption promoting agent, water, solvent, preservative, surfactant, and a pH balancer.

49. The topical composition according to claim 48, wherein said transdermal permeation enhancer is propylene glycol, Azone, or a combination thereof.

50. The topical composition according to claim 49, wherein said propylene glycol or Azone is present at a concentration of about 2% by weight.

51. The topical composition according to claim 48, further comprising alcohol and/or a PBS solution.

52. The topical composition according to claim 51, wherein said PBS solution has a pH at from about 6.5 to about 7.8.

53. The topical composition according to claim 42, wherein said composition is in the form of a gel, liniment, cream or ointment.

54. A method of reducing or delaying hair loss, or of increasing or promoting hair growth in a subject, the method comprising administering to said subject a topical composition of claim 1 in an amount effective to reduce or delay hair loss, or to increase or promote hair growth.

55. The method according to claim 54, wherein said hair loss is caused by androgenetic alopecia, or seborrheic alopecia.

56. The method according to claim 54, wherein said hair growth is eye brow or eye lash growth.

57. The method according to claim 54, wherein said hair loss or growth is related to PG E/F2a release.


SEQUENCE LISTING (2 sequences)

SEQ ID NO: 1 - protein, 28 aa, Homo sapiens
SSCTVAETERRATVKRFRKKSWEVYQGV

SEQ ID NO: 2 - protein, 40 aa, Homo sapiens
SSCTVAEDRYLALVKTMSMGRTERRATVLVRFRKKSWEVY